(19) United States
(12) Patent Application Publication
(10) Pub. No.: US 2009/0156656 A1
(43) Pub. Date: Jun. 18, 2009

(54) USE OF LIGHT-ACTIVE INDOCYANINE GREEN FOR TREATING INFLAMMATORY DISORDERS IN THE ORAL CAVITY

(75) Inventors: Olaf Schafer, Singen (DE); Daniela Krejci, Commugny (CH); Martin Werner Klarenaar, Stockach (DE)

Correspondence Address: BACHMAN & LAPOINTE, P.C., 900 CHAPEL STREET, SUITE 1201, NEW HAVEN, CT, 06510, US

(73) Assignee: ELEXXION GmbH, Radolfzell (DE)

(21) Appl. No.: 12/281,995

(22) PCT Filed: Mar. 5, 2007
(86) PCT No.: PCT/EP07/01867

§ 371 Date: Nov. 25, 2008

(30) Foreign Application Priority Data
Mar. 8, 2006 (DE) 10 2006 011 043.9

(51) Int. Cl. A61K 31/403; A61P 29/00

(52) U.S. Cl. 514/411

(57) ABSTRACT

In a process for treating disorders, especially inflammatory disorders, in the oral cavity, by introducing a substance activable by a light source, the substance comprises the active ingredient indocyanine green and the spectrum of the light source emits significant proportions in the range of 700-1000 nm.

### BACKGROUND OF THE INVENTION

The invention relates to a method for treating in particular inflammatory disorders in the oral cavity by inserting a substance activable by a light source.

Photodynamic therapy is a photochemical method which was previously used mainly in cancer therapy. This term is generally understood to mean a light induced deactivation of cells, microorganisms or molecules. An active ingredient attaches itself to the cells to be fought and is then activated by light.

A modification of this principle is used to fight microorganisms in antimicrobial photodynamic therapy (APT). Here the goal is not the destruction of the body's (tumor) cells, but the targeted fighting of local infections, i.e. the fighting of microorganisms. The principle of action of the APT is based on selective dyeing of the microorganisms by means of a so-called photosensitizer and destroying the germs by illumination using a suitable light source matched to the photosensitizer.

A corresponding method is disclosed in WO 01/87416 A1. The microorganisms are sensitized and/or dyed with the aid of the substance activable by light, in particular a dye, and are killed after irradiation with light of a suitable wavelength and energy density: the energy generated by means of an irradiation unit, in particular a laser unit, is concentrated onto the microorganisms therewith, and the balanced states of reactions, which also occur in the non-illuminated state in the “normal” environment, are displaced and as a consequence the microorganisms are destroyed.

Furthermore, EP 0 637 976 B1 discloses the use of a photosensitizing substance or compound, or a photosensitizer (PS), in medicine production for use in disinfecting or sterilizing tissues of the oral cavity or a wound or lesion in the oral cavity by destroying microbes which are related to a disease and found in a periodontal pocket, i.e. in the region between the tooth and the gingiva. Here, the photosensitizer touches the tissues, wound or lesion, as a result of which the microbes related to the disease take up the photosensitizer. The tissues, wound or lesion is or are irradiated by laser light at a wavelength absorbed by the photosensitizer. The reduction of germs by this combined laser-dye treatment is described for various germs and photosensitizers in the form of solutions comprising inter alia methylene blue and toluidine blue, with the influence of the applied energy density also being presented. HeNe lasers and GaAs lasers are used as light sources.

It is an object of the present invention to develop a method of the above-mentioned type in which a suitable active ingredient is used which in particular can no longer be detected even after a short period of time.

### SUMMARY OF THE INVENTION

The object is achieved due to the fact that the substance comprises indocyanine green as an active ingredient and a significant proportion of the spectrum of the light source is emitted in the 700-1000 nm range.

### DETAILED DESCRIPTION

Indocyanine green is an infrared dye which is used in medical diagnostics and utilized as a laser dye under the name IR 125. As an intravenous diagnostic active ingredient with a broad spectrum, it is used in cardiovascular, microcirculation and liver function diagnostics, and in particular also in measurements of the eye or retina.

According to the present invention, a completely new unexpected field of application is now disclosed, to be precise the use for treating in particular inflammatory disorders in the oral cavity together with an activating light source. The substance has the significant advantage that it can no longer be detected after approximately three hours.

It is intended that in particular semiconductor lasers (diode lasers) or light emitting diodes (LEDs) are used as a light source. The light sources used should preferably emit light with a wavelength in the 780-830 nm range. The output power of the light source is preferably in the 50 mW-5000 mW range. This results in a radiation dose of between 0.5 J and 50 J in the case of an illumination duration of 10 s-100 s.

It is intended that when the light is applied the light source is pulsed in the 1000-50,000 Hz range, preferably in the 10,000 Hz-20,000Hz range. In the process, the break between two pulses can be longer than the duration of the pulse.

The substance is administered in a sterile dosage form. Furthermore, it is possible that further ingredients which have a positive effect on inflammatory processes are added to the substance.

The dosage form lends itself to the substance remaining in the oral cavity for a certain amount of time in the form of a mouth rinse and the active ingredient being illuminated by a light source after the active ingredient has been rinsed out. Here the active ingredient is illuminated by a device which comprises the light source and which is carried temporarily (e.g. during a work break or overnight). It is also possible to insert the active ingredient solution via an appropriate administering device, such as a syringe, into areas which are difficult to reach, such as periodontal pockets or the like.

1-14. (canceled)

15. A method for treating inflammatory disorders in an oral cavity comprising the steps of:
providing a substance comprising at least indocyanine green as an active ingredient;
administering the substance in an aqueous mouth rinse in the oral cavity; and
activating the substance with a light source.

16. The method as claimed in claim 15 wherein the aqueous mouth rinse receives a radiation dose of between 0.5 J and 50 J in the case of an illumination duration of 10 to 100 s.

17. The method claimed in claim 15 wherein the substance remains in the oral cavity for a certain amount of time in the form of a mouth rinse and the active ingredient is illuminated by the light source after the active ingredient has been rinsed out.

18. The method as claimed in claim 17 wherein the active ingredient is illuminated by a device which comprises the light source.

19. The method as claimed in claim 15 wherein one of semiconductor lasers and light emitting diodes are used as a light source.

20. The method as claimed in claim 19 wherein the output power of the light source is in the 50 mW-5000 mW range.

21. The method as claimed in claim 20 wherein at the frequency of the light source, the break between two pulses is longer than the duration of the pulse.

22. The method as claimed in claim 15 including further ingredients which have a positive effect on inflammatory processes are added to the substance.

23. The method as claimed in claim 15 including administering the substance in a sterile dosage form.
